(19) United States
(12) Patent Application Publication
(10) Pub. No.: US 2024/0382182 A1
(43) Pub. Date: Nov. 21, 2024

(54) ULTRASONIC DOPPLER FLOW IMAGING METHOD

(71) Applicant: Shantou Institute of Ultrasonics Instruments Co, Ltd., Shantou (CN)

(72) Inventors: Liexiang FAN, Shantou (CN); Zhongyun WEI, Shantou (CN); Bin LI, Shantou (CN); Yuqiang KANG, Shantou (CN); Haomiao QIU, Shantou (CN); Yu WANG, Shantou (CN); Zehang CAI, Shantou (CN); Zhonghong WU, Shantou (CN); Peifeng CHEN, Shantou (CN)

(73) Assignee: Shantou Institute of Ultrasonic Instruments Co., Ltd., Shantou (CN)

(21) Appl. No.: 18/746,620

(22) Filed: Jun. 18, 2024

Related U.S. Application Data
(63) Continuation of application No. PCT/CN2023/094662, filed on May 17, 2023.

(30) Foreign Application Priority Data
May 16, 2023 (CN) 202310547855.5

(51) Int. Cl. A61B 8/08; A61B 8/06

(52) U.S. Cl. CPC A61B 8/5269; A61B 8/06; A61B 8/488

(57) ABSTRACT

Disclosed is an ultrasonic Doppler flow imaging method. The method includes: using eigen decomposition on an ultrasonic image to obtain eigenvectors matrices with different power values, so that the SNR and power distribution of each point of the ultrasonic image are calculated, thereby accurately extracting flow data with high reliability and obtaining accurate and stable flow images. The method has the beneficial effects of filtering out tissue signals and noise signals on the characteristic dimension, so as to better extract flow data by using eigen decomposition on ultrasonic images, retaining complete flow data and providing stable and clear imaging of fine flow without the need of contrast agent injection that may cause injuries to human bodies.

### CROSS-REFERENCE TO RELATED APPLICATIONS

The present application is a continuation application of International Application No. PCT/CN2023/094662, filed on May 17, 2023, which claims priority to Chinese Patent Application No. 202310547855.5, filed on May 16, 2023. All of the aforementioned applications are incorporated herein by reference in their entireties.

### TECHNICAL FIELD

The application relates to the field of ultrasonic image processing, in particular to an ultrasonic Doppler flow imaging method.

### BACKGROUND

Traditional color Doppler ultrasound has its limitations in observing small vessels and slow blood flow, in particular to parenchymal organs where tissue and flow information are superimposed. In conventional flow imaging processing, a wall filter, generally a high-pass filter, is used to filter out tissue signals. When the flow velocity is low, the frequency of tissue and flow is close, and the conventional wall filter cannot separate flow signals completely, thereby the acquired flow signals are discontinuous and unstable. To solve this problem, contrast agent is usually injected into the vessels to enhance the reflections, with an aim to improving the accuracy of flow detection, which is an invasive approach.

### SUMMARY

The application aims to provide an ultrasonic Doppler flow imaging method, and particularly provides an ultrasonic Doppler imaging method which is noninvasive that can obtain accurate, complete, clear and stable flow images.

In order to achieve this objective, the application adopts the technical solution below, an ultrasonic Doppler flow imaging method comprising the following steps:
S01, using eigen decomposition on an ultrasonic image from ultrasound scan to obtain N eigenvectors matrices, and enveloping data of the eigenvectors matrices to obtain power of the eigenvectors matrices, which are: high-power eigenvectors matrices, medium-power eigenvectors matrices and low-power eigenvectors matrices respectively; wherein, the N/5 eigenvectors matrices with the highest power values are selected from the N eigenvectors matrices as “high-power eigenvectors matrix”, the N/10 eigenvectors matrices with the lowest power values are selected from the N eigenvectors matrices as “low-power eigenvectors matrix”, and the rest are “medium-power eigenvectors matrix”;
S02, determining the point distribution of the ultrasound image in each eigenvectors matrix, and retaining the points mainly distributed in the medium-power eigenvectors matrices based on the data of the N eigenvectors matrices obtained in step S01;
S03, taking the data of the low-power eigenvectors matrices in step S01 as noise signals, and calculating the signal-to-noise ratio (SNR) of each point of the ultrasonic image. The points with SNR lower than the set value are considered as points with low reliability and eliminated, and the points with SNR higher than the set value are considered as points with high reliability and retained;
S04, combining the results of steps S02 and S03, extracting the points complying with main distribution in the medium-power eigenvectors matrices in step S02 and high reliability in step S03, thereby obtaining flow image data with high reliability;
Specifically, the method further comprises step S05, processing the reliable flow image data by using the opening and closing function with an X-Shape element for reconnecting the discontinuous parts and rejecting background noise, so as to obtain continuous clear flow image data;
Specifically, the method further comprises step S06, calculating the continuous clear flow image data from step S05 for its area of connected components, with the points where the area of connected components less than the set value regarded as singularity point, and obtaining continuous clear and noise-free flow image data after eliminating the singularity point.

The method has the beneficial effects that tissue signals and noise signals can be filtered out on the characteristic dimension so as to better extract flow data by using eigen decomposition on the ultrasonic image, so that complete retention of flow data is realized, stable and clear imaging of fine flow can be performed, without the harm from the contrast agent injected to a human body.

### BRIEF DESCRIPTION OF THE DRAWINGS

FIG. 1 is a flowchart of an ultrasonic Doppler flow imaging method in the embodiment.

### DETAILED DESCRIPTION OF THE EMBODIMENTS

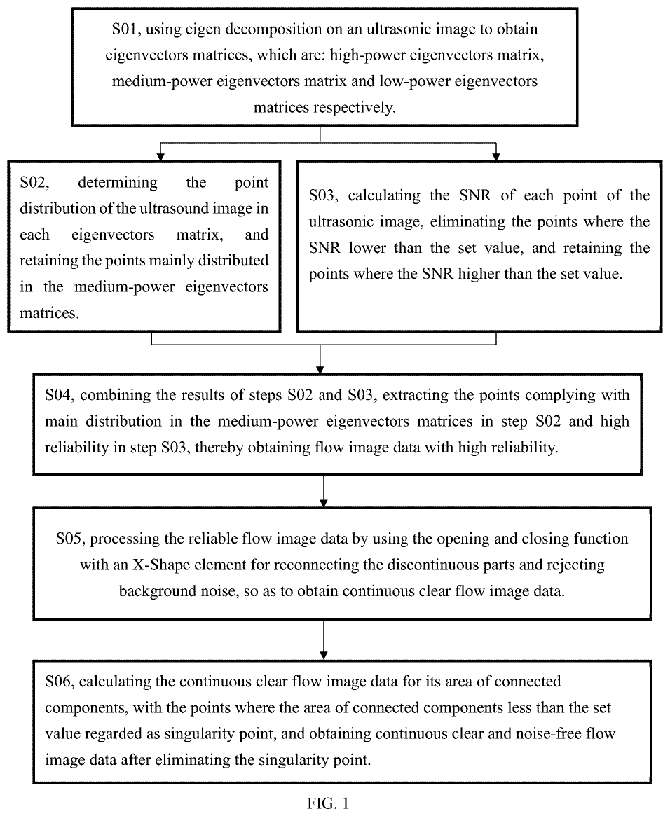

Embodiment 1, referring to FIG. 1, is an ultrasonic Doppler flow imaging method comprising the following steps:
S01, using eigen decomposition on an ultrasonic image from ultrasound scan to obtain N eigenvectors matrices, and enveloping data of the N eigenvectors matrices to obtain power of the eigenvectors matrices, which are: high-power eigenvectors matrices, medium-power eigenvectors matrices and low-power eigenvectors matrices respectively; wherein, the N/5 eigenvectors matrices with the highest power values are selected from the N eigenvectors matrices as “high-power eigenvectors matrix”, the N/10 eigenvectors matrices with the lowest power values are selected from the N eigenvectors matrices as “low-power eigenvectors matrix”, and the rest are “medium-power eigenvectors matrix”. The high-power eigenvectors matrices is for tissue signals, the medium-power eigenvectors matrices for flow signals and the low-power eigenvectors matrices for noise signals. It should be noted that in the above selection of the high-power eigenvectors matrices and the low-power eigenvectors matrices, N/5 and N/10 should both be integers, and in case the result is not an integer, the calculation is performed in a downward rounding manner;
S02, determining the point distribution of the ultrasound image in each eigenvectors matrix, and retaining the points mainly distributed in the medium-power eigenvectors matrices based on the data of the N eigenvectors matrices obtained in step S01;
S03, taking the data of the low-power eigenvectors matrices in step S01 as noise signals, and calculating the SNR of each point of the ultrasonic image. The points with SNR lower than the set value are considered as points with low reliability and eliminated, and the points with SNR higher than the set value are considered as points with high reliability and retained; and
S04, combining the results of steps S02 and S03, extracting the points complying with main distribution in the medium-power eigenvectors matrices in step S02 and high reliability in step S03, thereby obtaining flow image data with high reliability.

In addition, due to low contrast in micro flow, the method further comprises step S05, processing the reliable flow image data by using the opening and closing function with an X-Shape element for reconnecting the broken parts and rejecting background noise, so as to obtain continuous clear flow image data.

Moreover, to remove isolated noise points in the image, the method further comprises step S06, calculating the continuous clear flow image data from step S05 for its area of connected components, with the points where the area of connected components less than the set value regarded as singularity point, and obtaining continuous clear and noise-free flow image data after eliminating the singularity point.

Certainly the embodiments above are preferred for the present application only, but not intended to restrict the scope of use of the present application. Therefore, any equivalent changes made on the principles of the present application should be included in the protection scope of the present application.

What is claimed is:

1. An ultrasonic Doppler flow imaging method, comprising:
S01, using eigen decomposition on an ultrasonic image from ultrasound scan to obtain N eigenvectors matrices, and enveloping data of the N eigenvectors matrices to obtain power of the N eigenvectors matrices, which are: high-power eigenvectors matrices, medium-power eigenvectors matrices and low-power eigenvectors matrices respectively; wherein, the N/5 matrices with highest power values are selected from the N eigenvectors matrices as high-power eigenvectors matrix, the N/10 eigenvectors matrices with the lowest power values are selected from the N eigenvectors matrices as low-power eigenvectors matrix, and the rest are “medium-power eigenvectors matrix”;
S02, determining the point distribution of the ultrasound image in each eigenvectors matrix, and retaining the points mainly distributed in the medium-power eigenvectors matrices based on the data of the N eigenvectors matrices obtained in step S01;
S03, taking the data of the low-power eigenvectors matrices in step S01 as noise signals, and calculating a signal-to-noise ratio (SNR) of each point of the ultrasonic image, the points with SNR lower than the set value are considered as points with low reliability and eliminated, and the points with SNR higher than the set value are considered as points with high reliability and retained; and
S04, combining the results of steps S02 and S03, extracting the points complying with main distribution in the medium-power eigenvectors matrices in step S02 and high reliability in step S03, thereby obtaining flow image data with high reliability.

2. The ultrasonic Doppler flow imaging method according to claim 1, further comprising step S05, processing the reliable flow image data by using the opening and closing function with an X-Shape element for reconnecting the broken parts and rejecting background noise, so as to obtain continuous clear flow image data.

3. The ultrasonic Doppler flow imaging method according to claim 2, further comprising step S06, calculating the continuous clear flow image data from step S05 for an area of connected components, with the points where the area of connected components less than the set value regarded as a singularity point, and obtaining continuous clear and noise-free flow image data after eliminating the singularity point.
